(19) United States
(12) Patent Application Publication
(10) Pub. No.: US 2017/0167821 A1
(43) Pub. Date: Jun. 15, 2017

(54) Apparatus for launching and indicating speed of projectiles impelled by the force of breath

(71) Applicant: Chani C Martin, Portage, MI (US)

(72) Inventor: Chani C Martin, Portage, MI (US)

(21) Appl. No.: 14/965,326

(22) Filed: Dec. 10, 2015

(51) Int. Cl. F41B 1/00; A63B 71/06; A61B 5/09; A61B 5/087; A61B 5/00

(52) U.S. Cl. CPC F41B 1/00; A61B 5/0871; A61B 5/0875; A61B 5/742; A61B 5/09; A63B 71/0686; A63B 71/06

(57) ABSTRACT

A combined blow gun and projectile wherein the blowgun includes an elongated tubular component, a mouth piece, at least one sensor, and a visual indicator for projectile speed. The kit is simple and provides a method of qualitatively evaluating breath force whether for sport or for medical purposes.

### BACKGROUND

This disclosure relates generally to toys for children and more particularly to blowguns and peak flow meters.

A projectile, a small spherical object, is loaded into one end of a blowgun or peashooter and blown forcefully through by the user. This is a competitive sport especially for children and it would be useful to develop an improved version of the device.

### SUMMARY

One embodiment is an apparatus comprising an elongated tubular component, a mouth piece connected to the tubular component for application of air pressure, a generally spherical projectile concentric to the tubular component, at least one sensor attached to the tubular component, and a visual indicator connected to the sensor(s) providing a visual indication of speed of the projectile travelling through the tubular component.

Another embodiment is a method comprising obtaining an apparatus comprising an elongated tubular component, a mouth piece, a generally spherical projectile, and a velocity sensor configured to sense the velocity of the projectile traveling through the tubular component. The method also includes blowing through and ejecting the projectile and reading the projectile velocity output on a display.

### BRIEF DESCRIPTION OF THE DRAWINGS

FIG. 1 is a side view of a first embodiment of the present invention;

FIG. 2 is a perspective view of an apparatus with a form of indicators;

FIG. 3 is a cutaway view along the length of the apparatus depicted in FIG. 2;

FIG. 4 is a perspective view of a second embodiment of an indicator;

FIG. 5 is a cutaway view along the length of the apparatus depicted in FIG. 4;

FIG. 6 is a front view of a generic schematic version of the apparatus;

FIG. 7 is a side view of a generic schematic version of the apparatus;

FIG. 8 is a perspective view of a third embodiment of an indicator;

FIG. 9 is a perspective view of a fourth embodiment of an indicator;

FIG. 10 is a cutaway view along the length of another embodiment of the apparatus;

FIG. 11 is a view of a gripping portion of another embodiment of the apparatus;

FIG. 12 is a block diagram of electronic components in the apparatus;

### DETAILED DESCRIPTION

One embodiment described herein is a blowgun and projectile combination wherein a sensor and display combination tracks the speed of an impelled object. In embodiments, the blowgun is primarily made of a plastic safe for children to interact and play with. In embodiments, the projectile is primarily a sphere created from any range of materials including plastic, foam, rubber, or wood. The combination is low cost and offers use as either a children's toy or a fun alternative form of a peak flow meter for young children.

The embodiments of a blowgun described here are configured to display the speed of projectiles impelled through the tube by the force of breath. The speed may be displayed in a variety of forms in order to give the user a way to quantitatively measure their skill. In embodiments, the user can directly measure and display the velocity of projectile going through the blowgun, allowing data to be acquired from use. This can be used as a toy by children in a competitive nature. Another use is in order to measure breathing for asthmatic children who need to measure out medication accordingly.

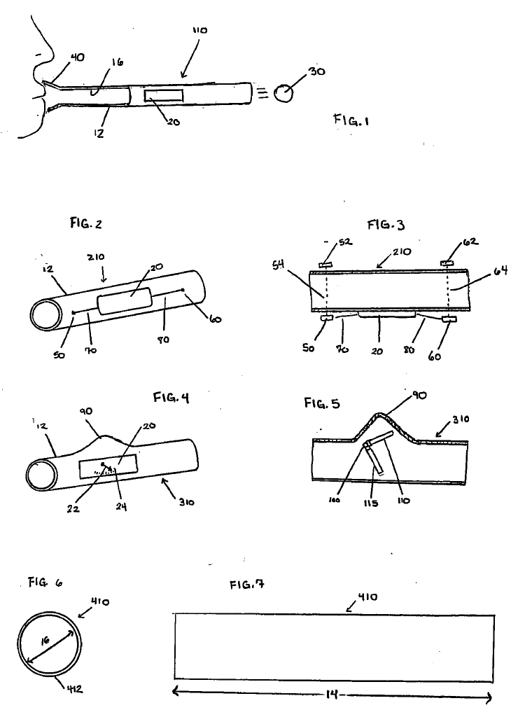

Referring to the drawings, FIG. 1 shows apparatus 110. The rendering is not to scale. For clarity, a cutaway of the front portion of the apparatus is shown. The tube 12 has a thin wall with a hollow inside surface 16. A flanged opening 40 may facilitate use as a mouth opening. Indicator display 20 may be placed upon tube 12.

Projectile 30 has a diameter slightly smaller than the inside wall 12 to allow easy movement. Projectile 30 is preferably not much smaller than inside wall 12 to prevent blown air from escaping. The tube and projectile can be packaged as a kit.

Now referring to FIGS. 2 and 3, apparatus 210 incorporates a device configured to sense velocity for the indicator display 20. On one side of tube 12 break beam emitters 52 and 62 are mounted. The emitters 52 and 62 emit a form of light or laser 54 and 64. The receivers 50 and 60 are mounted on the other side of tube 12 and detect emitted waves. An electronic timing device in receivers 50 and 60 relays information to through wires 70 and 80. Processed information is displayed through display 20. As shown in FIG. 3, the break beams 54 emit through the middle of tube 20 in order for projectile 30 to pass through and break beams 54 and 64.

Now referring to FIGS. 4 and 5, apparatus 310 shows an alternate method of sensing and indication. A protrusion 90 in tube 12 allows for movement of a moveable vane 120, and optionally at least one additional moveable vane 115, connected to a rotating axle 100. As air is impelled through tube 12, the vanes 120 and 115 rotate axle 100 thereby rotating indicator arrow 22. Axle 100 runs entirely through tube 12 to the outer side. Indicator arrow 22 moves along indicator display 24 displaying the speed corresponding to the force used to blow and rotate vane 115.

Now referring to FIGS. 6 and 7, the generic schematic 410 shows inside diameter 16 of tube 412 and length 14 with dimensions optimized for allowing a projectile to propel through by use of breath. Length 14 and diameter 16 may vary accordingly to projectile type and use case. In embodiments, a length 14 to diameter 16 ratio can be in the range of about 5:1 to about 20:1, or about 10:1 to about 15:1. In embodiments, the length to diameter ratio is about 12:1.

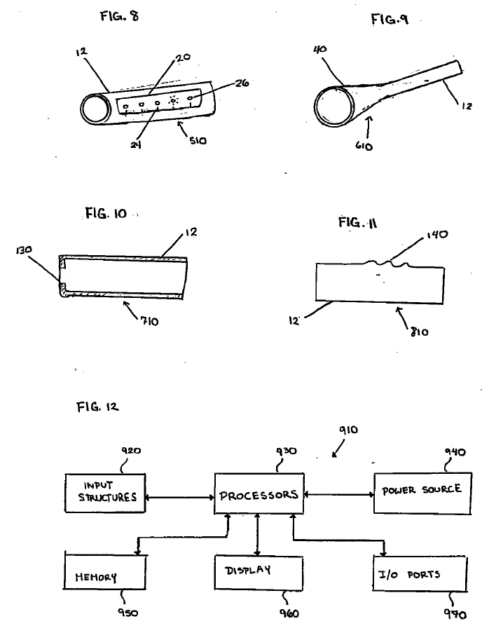

Various features and embodiments that can be incorporated into the embodiments shown in FIGS. 1-5 are illustrated in FIGS. 8-11. FIG. 8 shows an alternate form of indicating for apparatus 510. Tube 12 holds the LED 26 on display 20 according to markings 24 placed linearly to correspond to speed output. In FIG. 9, apparatus 610 is used to show tube 12 in a configuration utilizing frustoconical mouthpiece 40. The mouthpiece 40 can refer to a built-in feature as part of tube 12 or as a separate piece attached to the tube by other means. In FIG. 10 apparatus 710 is shown in a cutaway view to illustrate the use of integrating a safety stopper 130 into tube 12. Stopper 130 is a way to prevent a projectile from falling back in towards the user. In FIG. 11, apparatus 810 has molded gripping features 140 built into tube 12 to allow easy grasp and use while impelling breath.

FIG. 12 illustrates a block diagram 910 of components that may be present in the apparatus. The components in diagram 910 may be embodied by hardware and/or software components in the system. Components may include a processor or processors 930, input structure 920, power source 940, memory 950, display 960, and I/O ports 970. Input structure 920 includes a sensor array to detect the projectile's travel. The power source 940 can be a small battery that can provide the required power to the rest of the system. The memory 950 can the electronic component that allows for the temporary or permanent storage of data about the projectile's speed. The display 960 correlates to forms of display as mentioned in other embodiments. I/O ports 970 embody the communication methods between the computing components.

In embodiments, a method of using the apparatus includes using the force of breath through the mouthpiece to propel the projectile and determining the velocity of the projectile by looking at the indicator.

In embodiments, the tube has a length in the range of about 4 inches to about 18 inches. In embodiments, the tube has an inner diameter of the range of about ¼ inch to about 2 inches. The tube typically has a thickness of about ⅛ inch to about ¼ inch. In embodiments, the apparatus is typically made of a thermoplastic material, a thermoset material, or wood. In embodiments, the projectile is typically made of a thermoplastic material, a thermoset material, foam, or wood. In embodiments, the projectile is in a weight range allowing human breath to impel the projectile a sufficient distance.

Although the present apparatus has been described in considerable detail with reference to certain preferred versions thereof, other versions would be readily apparent to those of ordinary skill in the art. Therefore, the spirit and scope of the description should not be limited to the description of the versions contained herein. A number of alternatives, modifications, variations, or improvements therein may be subsequently made by those skilled in the art, which are also intended to be encompassed by the following claims.

What is claimed is:

1. An apparatus comprising:
an elongated tubular component;
a mouth piece connected to the tubular component for application of air pressure;
a generally spherical projectile concentric to the tubular component;
at least one sensor attached to the tubular component; and
a visual indicator connected to the sensor(s) providing a visual indication of speed of the projectile travelling through the tubular component.

2. The apparatus of claim 1, wherein said at least one sensor comprises a first trigger along a first portion of the tubular component, a second trigger along a second portion of the tubular component, a timer connected to the first and second triggers, and a processor providing output to the visual indicator from the timer.

3. The apparatus of claim 1, wherein said at least one sensor comprises a thin, flexible rotating member comprising a mounting portion rigidly fixed to the tubular component, a protrusion built into the tubular component to house the rotating member, and a physical indicator fixed to the rotating member external to the tubular component.

4. The apparatus of claim 2, wherein said visual indicator comprises a linear multiplicity of lights.

5. The apparatus of claim 1, wherein said mouthpiece comprises a frustoconical neck.

6. The apparatus of claim 1, wherein a safety stopper is proximate to the mouthpiece or in the mouthpiece.

7. The apparatus of claim 1, wherein the tubular component includes a gripping portion.

8. A method comprising:
obtaining an apparatus comprising an elongated tubular components, a mouth piece, a generally spherical projectile, and a velocity sensor configured to sense the velocity of the projectile traveling through the tubular component;
blowing through and ejecting the projectile; and
reading the projectile velocity output on a display.

9. The method of claim 8, wherein reading comprises reading an electronic display.

10. The method of claim 8, wherein reading comprises reading a non-digital display.
